(19) United States
(12) Patent Application Publication
(10) Pub. No.: US 2010/0247649 A1
(43) Pub. Date: Sep. 30, 2010

(54) PHARMACEUTICAL FORMULATIONS COMPRISING TELMISARTAN AND HYDROCHLOROTHIAZIDE

(75) Inventors: Uma Devi Palaparthi, Hyderabad (IN); Ashutosh Rout, Dhenkanal District (IN); Maheswara Reddy Arumalla, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500016 Andhra Pradesh, AP (IN); DR. REDDY'S LABORATORIES, INC., Bridgewater, NJ (US)

(21) Appl. No.: 12/740,732

(22) PCT Filed: Oct. 30, 2008
(86) PCT No.: PCT/US2008/081714

§ 371 Date: Apr. 30, 2010

Related U.S. Application Data
(60) Provisional application No. 61/025,838, filed on Feb. 4, 2008.

(30) Foreign Application Priority Data
Oct. 30, 2007 (IN) 2464/CHE/2007

(51) Int. Cl. A61K 31/549; A61K 9/30; A61K 9/10; A61K 9/14; A61P 9/12

(52) U.S. Cl. 424/475; 424/484; 424/490; 514/223.5

(57) ABSTRACT

Pharmaceutical tablets comprising a first layer formulated for immediate release of telmisartan from a dissolving matrix and a second layer formulated for immediate release of hydrochlorothiazide from a dissolving matrix, methods for producing tablets and methods of use for treating hypertension.

### INTRODUCTION

The present invention relates to pharmaceutical formulations comprising telmisartan, including any salt thereof, and hydrochlorothiazide. The present invention further relates to stable pharmaceutical formulations comprising telmisartan or a salt thereof and hydrochlorothiazide. The invention also relates to processes for preparing formulations and methods of using the formulations for treating hypertension.

Telmisartan has chemical names 4′-[(1,4′-dimethyl-2′-propyl[2,6′-bi-1H-benzimidazol]-1′-yl)methyl]-[1,1′-biphenyl]-2-carboxylic acid, or 2-[4-[[4-methyl-6-(1-methylbenzoimidazol-2-yl)-2-propyl-benzoinidazol-1-yl]methyl]phenyl]benzoic acid. Its empirical formula is C₃₃H₃₀N₄O₂, its molecular weight is 514.63, and it has structural Formula I.

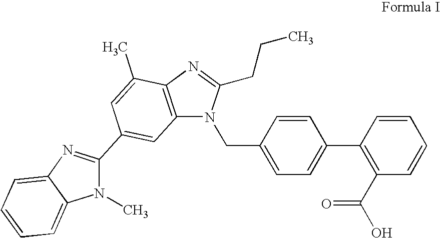

Telmisartan is a white to slightly yellowish solid. It is practically insoluble in water and in the pH range of 3 to 9, is sparingly soluble in strong acids (except insoluble in hydrochloric acid), and is soluble in strong bases.

Hydrochlorothiazide (sometimes abbreviated “HCT” or “HCTZ”) has chemical names 6-chloro-3,4-dihydro-2H-1,2,4-benzothiadiazine-7-sulfonamide 1,1-dioxide, or 6-chloro-2H-1,2,4-benzothiadiazine-7-sulfonamide 1,1-dioxide. Hydrochlorothiazide is a white, or practically white, practically odorless, crystalline powder with a molecular weight of 297.74. It is slightly soluble in water, and freely soluble in sodium hydroxide solution. Its empirical formula is C₇H₈ClN₃O₄S₂, and it has structural Formula II.

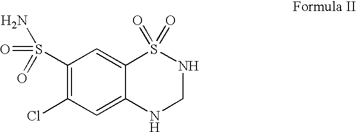

The commercially available product MICARDIS® HCT (sold by Boehringer Ingelheim, U.S.A.) for oral administration is available in three strengths, telmisartan/hydrochlorothiazide 80 mg/25 mg, 80 mg/12.5 mg, and 40 mg/12.5 mg. MICARDIS® HCT is indicated for the treatment of hypertension. Inactive ingredients are sodium hydroxide, meglumine, povidone, sorbitol, magnesium stearate, lactose monohydrate, microcrystalline cellulose, maize starch, sodium starch glycolate, and a coloring agent that is either ferric oxide red or ferric oxide to yellow. Similar products are sold outside the U.S. as MICARDIS PLUS®. These products are formulated as bi-layer tablets, the telmisartan layer having a dissolving matrix and the hydrochlorothiazide layer having a disintegrating matrix.

Both diuretics and angiotensin II receptor antagonists have an effect on the renin-angiotensin-aldosterone system. Angiotensin II receptor antagonists lower blood pressure by blocking angiotensin II receptors, important in regulating the blood pressure. Diuretics regulate sodium balance and thereby also extracellular fluid volume, which results in decreases of both sodium and fluid volume.

U.S. Pat. No. 5,591,762 discloses benzimidazoles (including telmisartan) that are useful as angiotensin II antagonists. U.S. Pat. No. 6,358,986 discloses a mixture of two polymorphic crystalline forms of telmisartan.

U.S. Pat. No. 4,970,078 discloses a pharmaceutical tablet comprising hydrochlorothiazide and crosslinked carboxymethyl guar, the guar being crosslinked with HCl, guar comprising 1 to 2% by weight of the tablets and the guar having a degree of substitution between 0.17 and 0.21.

U.S. Patent Application Publication No. 2004/0110813 discloses a pharmaceutical composition comprising 3-50 wt. percent telmisartan dispersed in a dissolving matrix comprising: (a) a basic agent in a molar ratio of basic agent to telmisartan of 1:1 to 10:1; (b) about 1 to about 20 wt. percent of a surfactant or emulsifier; (c) 25 to 70 wt. percent of a water-soluble diluent; and (d) 0 to 20 wt. percent of one or more additional excipients and/or adjuvants, wherein the sum of all components is 100%.

U.S. Patent Application Publication No. 2005/089575 discloses a bilayer pharmaceutical tablet comprising a first layer containing telmisartan in at least 90% amorphous form in a dissolving tablet matrix comprising a basic agent, and a second layer containing hydrochlorothiazide in a disintegrating tablet matrix.

U.S. Patent Application Publication No. 2006/0159747 discloses a pharmaceutical composition comprising about 80 mg of telmisartan; and about 25 mg of hydrochlorothiazide. U.S. Patent Application Publication No. 2008/0113023 and International Application Publication Nos. WO 2007/144175 and WO 94/09778 A1 disclose pharmaceutical formulations of telmisartan and hydrochlorothiazide.

It is known from the literature that telmisartan has very poor aqueous solubility in the physiological pH range of the gastrointestinal tract between pH 1 and 7, and is soluble in strong base. On the other hand, hydrochlorothiazide degrades in alkaline conditions. It is advantageous to administer both drugs concomitantly or even better to administer a composition comprising both, to treat hypertension. Difficulty results from the fact that both active substances need to be released from the composition substantially simultaneously.

Preparation of fixed dose combinations of telmisartan and hydrochlorothiazide with adequate stability poses a challenge to the formulator. Hence there is need for simple and stable fixed dose compositions containing telmisartan and hydrochlorothiazide.

### SUMMARY

The present invention relates to pharmaceutical formulations comprising telmisartan or a salt thereof and hydrochlorothiazide, processes for preparing the formulations, and methods of using such formulations for treating hypertension.

In a embodiment invention includes compositions comprising telmisartan or a salt and hydrochlorothiazide, which have immediate drug release characteristics.

The present invention further relates to pharmaceutical compositions of telmisartan and hydrochlorothiazide, wherein the contained telmisartan is less than about 90% amorphous.

In another embodiment the invention includes pharmaceutical formulations comprising telmisartan in a dissolving matrix and hydrochlorothiazide in a dissolving matrix, wherein the contained telmisartan is substantially amorphous in nature.

In an embodiment the invention includes pharmaceutical compositions comprising telmisartan and hydrochlorothiazide, wherein average particle sizes of the telmisartan used are about 1 to about 100 μm, or about 1 to about 50 μm, or about 1 to about 25 μm, and average particle sizes of the hydrochlorothiazide used are about 1 to about 100 μm, or about 1 to about 75 μm, or about 1 to about 50 μm.

In an embodiment, the invention includes stable pharmaceutical formulations comprising telmisartan or a salt thereof, and hydrochlorothiazide.

In another embodiment the invention includes pharmaceutical formulations comprising telmisartan or a salt thereof and hydrochlorothiazide, wherein the formulations may be in the forms of monolithic, or multilayered, or inlayered, or multiparticulate systems.

Further the invention relates to pharmaceutical formulations comprising telmisartan and hydrochlorothiazide in a bilayered form, wherein a first layer comprises telmisartan in a disintegrating matrix comprising a basic agent, and a second layer comprises hydrochlorothiazide in a dissolving matrix.

An aspect of the present invention includes formulations comprising compositions of telmisartan and hydrochlorothiazide in a bilayer tablet form, wherein both the layers are present in a disintegrating matrix.

Another embodiment of the present invention includes formulations comprising compositions of telmisartan and hydrochlorothiazide in a bilayer tablet form, wherein both the layers are present in a dissolving matrix.

A further embodiment of the present invention includes formulations comprising compositions of telmisartan and hydrochlorothiazide in tablet form, wherein telmisartan is present in a dissolving tablet matrix, which is in the form of a core, with a dissolving coat of hydrochlorothiazide applied onto the core.

Another embodiment of the present invention includes formulations comprising telmisartan and hydrochlorothiazide in tablet form, wherein both are present in multiparticulate systems and mixed together and compressed into tablets, or alternatively filled into capsules.

An aspect of the invention provides a pharmaceutical formulation comprising a first portion containing telmisartan in a dissolving matrix and a second portion containing hydrochlorothiazide in a dissolving matrix.

Another aspect of the invention provides a process for preparing a pharmaceutical bi-layer tablet comprising:

a) granulating a solid composition comprising a diluent with a solution comprising telmisartan, to provide a first tablet layer composition;

b) combining hydrochlorothiazide with at least one solid pharmaceutical excipient to provide a second tablet layer composition; and

c) compressing a first tablet layer composition and a second tablet layer composition into a bi-layer tablet.

### BRIEF DESCRIPTION OF THE DRAWING

FIG. 1 shows comparative powder X-ray diffraction (XRD) patterns for the formulation prepared according to Example 10, wherein “A” represents starting telmisartan, “P” represents a placebo formulation, omitting the telmisartan, “B” represents the formulation as initially prepared, and “C” represents the formulation after storage at 40° C. and 75% relative humidity for 1 month.

### DETAILED DESCRIPTION

The present invention relates to pharmaceutical formulations comprising compositions comprising telmisartan, including salts thereof, and hydrochlorothiazide. The present invention further relates to stable pharmaceutical formulations comprising telmisartan or salts thereof, and hydrochlorothiazide. The invention also relates to processes for preparing formulations and methods of using the formulations for treating hypertension.

In an embodiment the invention includes formulations comprising telmisartan or salts thereof and hydrochlorothiazide, which are for immediate release.

The present invention further relates to pharmaceutical formulations comprising telmisartan and hydrochlorothiazide, wherein contained telmisartan is less than about 90% amorphous.

As both telmisartan and hydrochlorothiazide are water-insoluble drugs, particle sizes of the drugs can play a role in the drug dissolution. The rate of dissolution of a poorly soluble drug is a rate-limiting factor in its absorption by the body. It is recognized that such drugs may be more readily bioavailable if administered in a finely divided state. Because of the poor water solubility of telmisartan and hydrochlorothiazide, the rate of dissolution of drug from a dosage form is a factor in determining the rate and extent of drug absorption. The rate of dissolution depends on factors including particle sizes (or particle surface areas, which can be related to particle sizes).

The percent of particles with different dimensions that exist in a powder is called the particle size distribution. It is represented in certain ways. Particle size is the maximum dimension of a particle, frequently expressed in units of μm. Particle size distributions can be expressed in terms of, D₁₀, D₅₀, D₉₀ and D_{[4,3]}. The D₁₀, D₅₀ and D₉₀ represent the 10th percentile, median or 50th percentile, and the 90th percentile of the particle size distribution, respectively, as measured by volume. That is, the D₁₀, D₅₀, and D₉₀ are values of the distribution such that 10%, 50%, and 90% by volume of the particles have a size of the stated value or less, or is the percentage of particles smaller than those sizes. D₅₀ is also known as the median, or average, size of particles. It is one of the important parameters representing characteristics of particles in a powder. For a sample, if D₅₀=5 μm, it means that 50% by volume of the particles are smaller than 5 μm. Similarly, if D₁₀=5 μm, 10% by volume of the particles are less than or equal to 5 μm, and if D₉₀=5 μm, 90% by volume of the particles are less than or equal to 5 μm. D_{[4,3]} is the volume moment mean of the particles or the volume weighted particle size. Particle size distribution can conveniently be determined using any of various types of equipment, such as laser light scattering instrumentation sold by Malvern Instruments Ltd, of Malvern, Worcestershire, United Kingdom.

The particle sizes of the telmisartan and hydrochlorothiazide for use in the present invention can be obtained by any suitable process and equipment known in the art, for example process such as sieving and equipment such as air jet mills, pulverizers, and fluid energy mills.

In an embodiment the invention includes pharmaceutical formulations comprising telmisartan and hydrochlorothiazide, wherein average particle sizes of the telmisartan used are about 1 to about 100 μm, or about 1 to about 50 μm, or about 1 to about 25 μm, and average particle sizes of the hydrochlorothiazide used are about 1 to about 100 μm, or about 1 to about 75 μm, or about 1 to about 50 μm.

In an embodiment of the invention the telmisartan used to prepare formulations is less than about 90% amorphous.

In an embodiment the invention relates to pharmaceutical formulations comprising telmisartan and hydrochlorothiazide, wherein the contained telmisartan is less than about 90% amorphous.

In an embodiment the invention includes pharmaceutical formulations comprising telmisartan in a dissolving matrix and hydrochlorothiazide in a dissolving matrix, wherein the contained telmisartan is substantially amorphous.

It has been found that telmisartan has pH-dependant solubility and is soluble in strongly alkaline conditions. Also, hydrochlorothiazide has been observed to be sensitive to alkaline conditions. Therefore, for compositions comprising telmisartan and hydrochlorothiazide, if a strong alkalizing agent is present, bi-layer tablet technology has been adopted to provide stable formulations comprising telmisartan and hydrochlorothiazide.

In an embodiment, telmisartan is present in a dissolving matrix in core tablets, and a coating solution that contains hydrochlorothiazide is applied onto the cores; by this technique, the composition forms a bi-layer system in which telmisartan is present in dissolving cores and hydrochlorothiazide is present in dissolving coatings.

In another embodiment, telmisartan and hydrochlorothiazide are separately formulated into multiparticulate systems and mixed together, and can be compressed into tablets or filled into capsules.

In all of the above embodiments, the telmisartan component of the formulation is associated with one or more basic agents, with or without a soluble diluent, and the hydrochlorothiazide component is associated with disintegrating or dissolving diluents.

In accordance with the invention, the term “dissolving matrix” refers to a composition dosage form which, when present in a testing fluid environment, would release the entire drug content into the fluid to form a reasonably clear solution. This infers that when the dosage form is in a physiological medium, the drug readily dissolves in it and enhances systemic absorption.

In accordance with the invention, the term “disintegrating matrix” refers to a composition of the dosage form which, when present in a testing fluid environment, would disintegrate into small particles, from which the drug can be released into the fluid.

In another embodiment the invention relates to pharmaceutical formulations comprising telmisartan or a salt, and hydrochlorothiazide, wherein the formulations may be in the forms of monolithic, multilayered, inlay, or multiparticulate systems.

Further the invention relates to pharmaceutical formulations comprising telmisartan and hydrochlorothiazide in a bi-layered form, wherein a first layer comprises telmisartan in a disintegrating matrix comprising a basic agent, and a second layer comprises hydrochlorothiazide in a dissolving matrix.

An embodiment of the present invention includes formulations comprising formulations comprising telmisartan and hydrochlorothiazide in a bi-layer tablet form, wherein both layers have a disintegrating matrix.

Another embodiment of the present invention includes formulations comprising telmisartan and hydrochlorothiazide in a bi-layer tablet form, wherein both layers have a dissolving matrix.

A further embodiment of the present invention includes formulations comprising telmisartan and hydrochlorothiazide in tablet form, wherein telmisartan is present in a dissolving tablet matrix, which is in the form of a core, with a dissolving coating of hydrochlorothiazide applied onto the core.

Another embodiment of the present invention includes formulations comprising telmisartan and hydrochlorothiazide in tablet form, wherein both drugs are present in multiparticulate systems and mixed together and compressed into tablets or alternatively filled into capsules.

Basifying agents that are useful in the compositions include, but are not limited to, alkali metal hydroxides such as sodium hydroxide and potassium hydroxide, carbonates such as sodium carbonate, sodium bicarbonate, potassium carbonate, and potassium bicarbonate, Na₂HPO₄ and K₂HPO₄, basic amino acids such as arginine and meglumine, etc.

In an embodiment, the invention includes stable pharmaceutical formulations comprising telmisartan or a salt thereof, and hydrochlorothiazide.

Hydrochlorothiazide degrades in alkaline conditions, i.e., undergoes alkaline hydrolysis in the presence of heat and moisture. A degradation product of hydrochlorothiazide is 4-amino-6-chloro-1,3-benzenedisulfonamide (“DSA”) represented by structural Formula III.

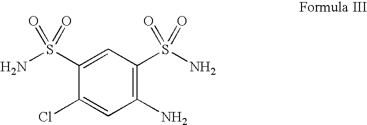

Other degradants of hydrochlorothiazide include chlorothiazide, represented by structural Formula IV, and 6-chloro-N-[(6-chloro-7-sulphamoyl-2,3-dihydro-4H-1,2,4-benzothiadiazin-4-yl 1,1-dioxide)methyl]-3,4-dihydro-2H-1,2,4-benzothiadiazine-7-sulphonamide 1,1-dioxide, represented by structural Formula V.

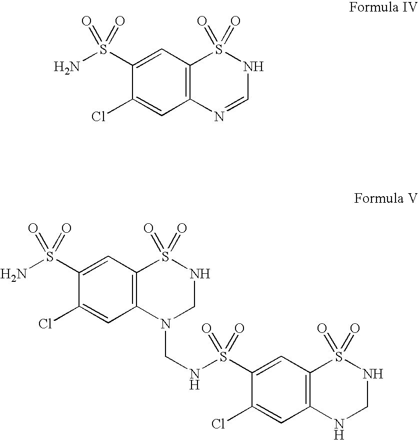

An impurity related to telmisartan is 2-n-propyl-4-methyl-6-(1-methyl benzimidazole-2-yl) benzimidazole (“TLA-2”) represented by structural Formula VI.

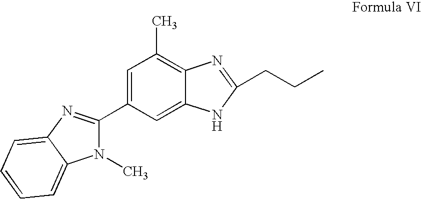

Another impurity related to telmisartan is [methyl-4′-[(2-n-propyl-4-methyl-6-(1-methyl benzimidazol-2-yl)-benzimidazol-1-yl)-methyl-biphenyl-2-carboxylate]hydrochloride (“TEL-2”) represented by structural Formula VII.

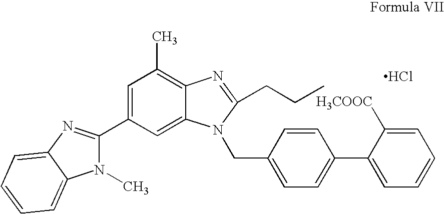

In an embodiment the invention includes stable pharmaceutical formulations comprising telmisartan or a salt thereof and hydrochlorothiazide.

In embodiments the invention includes stable pharmaceutical formulations comprising telmisartan or a salt thereof and hydrochlorothiazide, wherein the moisture content of the formulation is less than about 6% w/w.

In an embodiment the invention includes stable pharmaceutical formulations comprising telmisartan or a salt thereof and hydrochlorothiazide, wherein the DSA impurity is present at less than about 1% by weight of the label hydrochlorothiazide content.

In another embodiment the invention includes stable pharmaceuctical formulations comprising telmisartan or its salt and hydrochlorothiazide, wherein total impurities are present at less than about 4% by weight of the label total content of telmisartan and hydrochlorothiazide.

In an embodiment the invention includes analytical methods for the analysis of impurities, using high performance liquid chromatography (HPLC), wherein an embodiment of a method comprises the following:

Buffer solution: 0.7 g of sodium perchlorate dissolved in 1000 ml of water, with pH adjusted to 2.5 with dilute phosphoric acid. The solution can be filtered through a 0.45 μm nylon filter.

Mobile phase A: Buffer and acetonitrile in a volume ratio of 88:12.

Mobile phase B: Buffer and acetonitrile in a volume ratio of 25:75.

Diluent: 0.01N HCl and methanol (80:20 volume ratio).

Chromatographic System:

a) Liquid chromatograph equipped with a 271 nm UV detector.

b) Column is Capcell PAK, C18, 250×4.6 mm, 5 μm.

c) Temperature: 25° C.

d) Flow rate: 1 mL per minute.

e) Injection volume: 40 μL.

f) Run time: 80 minutes.

The relative retention times (RRT) of various impurities are tabulated below.

| | |
|---|---|
| Impurity | RRT |

Hydrochlorothiazide-related*

| | |
|---|---|
| DSA | 0.7 |
| Chorothiazide | 0.83 |

Telmisartan-related**

| | |
|---|---|
| TLA-2 | 0.57 |
| TEL-2 | 1.45 |

*Relative retention time, where hydrochlorothiazide = 1.
**Relative retention time, where telmisartan = 1.

Sample is prepared for analysis by placing tablet powder equivalent to about 20 mg of hydrochlorothiazide into a 200 ml volumetric flask, adding about 170 ml of diluent, and sonicating for 30 minutes at less than 25° C. The solution is then diluted to volume with diluent, and a portion is filtered through a 0.45 μm nylon filter before injection.

In an embodiment the present invention provides pharmaceutical formulations comprising telmisartan or any of its salts and hydrochlorothiazide, wherein the formulations are in solid oral dosage forms, such as tablets like minitablets, bilayered tablets, inlayered tablets, capsules, lozenges, pills, granules, etc.

The solid dosage forms or formulations may include any number of excipients, including, but not limited to, diluents or fillers, binding agents, disintegrants, coloring agents, lubricating agents, glidants, solvents, film-forming agents, and wetting agents.

Useful diluents include, but are not limited to, glucose, sucrose, lactose and sugar alcohols like mannitol, xylitol, and sorbitol.

Useful binders include, but are not limited to, dry binders and/or wet granulation binders, depending on the manufacturing process chosen to prepare the pharmaceutical composition. Suitable dry binders include cellulose powder, crystalline cellulose, microcrystalline cellulose, and light anhydrous silicic acid. Examples of wet granulation binders include polyvinylpyrrolidones (povidones), vinylpyrrolidone-vinyl acetate copolymers (copovidones) and cellulose derivatives like hydroxymethyl celluloses, hydroxyethylcelluloses, hydroxypropylcelluloses, and hydroxypropyl methylcelluloses.

Suitable disintegrants include sodium starch glycolate, crospovidone, croscarmellose, sodium carboxymethylcellulose and dried corn starch.

Other useful excipients and adjuvants include: diluents and carriers such as cellulose powder, crystalline cellulose or microcrystalline cellulose, cellulose derivatives like hydroxymethylcelluloses, hydroxyethylcelluloses, hydroxypropylcelluloses and hydroxypropyl methylcelluloses, dibasic calcium phosphate, pregelatinized starch, polyvinylpyrrolidones (povidones), etc.; lubricants such as stearic acid, magnesium stearate, sodium stearylfumarate, glycerol tribehenate, etc.; flow control agents such as colloidal silica, light anhydrous silicic acid, crystalline cellulose, talc, etc.; crystallization retarders such as povidones, etc.; coloring agents, including dyes and pigments such as iron oxide red or yellow, titanium dioxide, talc, etc.; and mixtures of any two or more of these excipients and/or adjuvants.

Various film-forming agents that are useful for coating dosage forms include but are not limited to cellulose derivatives such as soluble alkyl- or hydroalkyl-cellulose derivatives such as methyl celluloses, hydroxymethyl celluloses, hydroxyethyl celluloses, hydroxypropyl celluloses, hydroxymethylethyl celluloses, hydroxypropyl methylcelluloses, sodium carboxymethyl celluloses, etc., insoluble cellulose derivative such as ethyl celluloses and the like, dextrins, starches and starch derivatives, polymers based on carbohydrates and derivatives thereof, natural gums such as gum Arabic, xanthans, alginates, polyacrylic acid, polyvinyl alcohols, polyvinyl acetates, polyvinylpyrrolidones, polymethacrylates and derivatives thereof (Eudragit™ products), chitosan and derivatives thereof, shellac and derivatives thereof, waxes and fat substances. Useful enteric coating materials include but are not limited to materials such as cellulosic polymers like cellulose acetate phthalates, cellulose acetate trimellitates, hydroxypropyl methylcellulose phthalates, polyvinyl acetate phthalates, etc., methacrylic acid polymers and copolymers (Eudragit™), and the like, and mixtures thereof.

Some excipients are used as adjuvants for coating processes, including excipients such as plasticizers, opacifiers, antiadhesives, polishing agents, etc. Various useful plasticizers include but are not limited to castor oil, diacetylated monoglycerides, dibutyl sebacate, diethyl phthalate, glycerin, polyethylene glycols, propylene glycols, triacetin, triethyl citrate, and mixtures thereof. An opacifier like titianium dioxide may also be present in an amount ranging from about 10% (w/w) to about 20% (w/w) based on the total weight of the coating.

Anti-adhesives are frequently used in film coating processes to avoid sticking effects during film formation and drying. An example of an anti-adhesive for this purpose is talc.

Suitable polishing agents include polyethylene glycols of various molecular weights or mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax).

In addition to the above coating ingredients, sometimes pre-formulated coating materials such as OPADRY™ products (supplied by Colorcon) or TABCOAT™ products can be used. OPADRY compositions generally comprise polymer, plasticizer and, if desired, pigment in a dry concentrate. OPADRY products produce attractive, elegant coatings on a variety of tablet cores and can be used in both aqueous and organic coating procedures. Pre-mixed coating products generally require only dispersion in a liquid before use.

Further, the present invention includes processes for preparing formulations containing telmisartan and hydrochlorothiazide, wherein an embodiment of a process comprises:

1) sifting the active and excipients through a sieve;

2) dry mixing the active ingredient and suitable excipients, or only excipients;

3) optionally, blending the step 2 dry mix with a suitable lubricant;

4) optionally compacting the dry mix of step 2 and subjecting to milling;

5) granulating the dry mix of step 2 using an appropriate granulating fluid with or without active ingredient;

6) drying the wet mass of granules;

7) sizing the dried granules through a suitable sieve;

8) blending granules from step 4 or step 7 with suitable extragranular excipients; and

9) compressing the final blend of step 8 or from step 3 into tablets or alternatively filling into capsules.

Alternatively, active ingredient may be dissolved in a suitable solvent, optionally with suitable excipients, and may be coated onto suitable substrates. Active ingredient-coated particles are optionally mixed with suitable excipients to form a final blend.

Alternatively, one active ingredient-coated particle or tablet, comprising one active ingredient, may be further coated with another active ingredient dissolved in a suitable solvent, which forms a single particle comprising two active ingredients. Particles coated with a drug may be compressed or filled into capsules. Since the present invention involves a combination of two active ingredients, telmisartan and hydrochlorothiazide, the compositions for each active ingredient may be prepared using any of the above described processes. The composition (final blend) containing each active ingredient may be compressed together to form tablets, or alternatively together may be filled into capsules.

In another embodiment, the invention includes use of packaging materials such as containers including lids, composed of polyethylene and or polypropylene and/or glass, and blisters or strips composed of aluminium or high-density polypropylene or polyvinyl chloride, or polyvinyl chloride coated with polyvinylidene dichloride, generally termed as PVC/PVDC. Different grades of PVC/PVDC are available as PVC/PVDC 40 gsm, PVC/PVDC 60 gsm, PVC/PVDC 90 gsm, etc. PVC/PVDC 40 gsm means 40 grams of PVDC coating per square meter of PVC film. Similarly 60 gsm means 60 grams of PVDC coating per square meter of PVC film, 90 gsm means 90 grams of PVDC coating per square meter of PVC film, etc.

The formulations of the present invention may be prepared using operations including physical mixing, blending, dry granulation, wet granulation, direct compression, etc., and any combination of two or more of these.

Equipment suitable for processing the pharmaceutical compositions of the present invention includes rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans; tray dryers, fluid bed dryers, rotary cone vacuum dryers, and the like, multimills, fluid energy mills, ball mills, colloid mills, roller mills, hammer mills, and the like, and various sieves. All sieves that are used for processing the pharmaceutical compositions of the present invention are sized according to the standard ASTM International specifications.

Dosage forms can be subjected to in vitro dissolution testing, such as using the procedures according to Test 711 “Dissolution” in United States Pharmacopoeia 29, United States Pharmacopeial Convention, Inc., Rockville, Md., pages 2673-2682, 2005 (“USP”) to determine the rate at which active ingredient is released from the dosage forms, and content of active substance can be determined in dissolution media using analytical techniques such as high performance liquid chromatography. The media used for dissolution testing can vary, including water, surfactant solutions, and various fluids that correspond to conditions existing in the human digestive tract. The latter fluids generally range in pH values from about 1 to about 8, and include dilute hydrochloric acid, buffers having pH values of, for example, 4.5, 5.8, and 6.8, simulated gastric fluids containing pepsin, simulated intestinal fluids containing pancreatin, etc. The compositions of numerous different media that are useful for dissolution testing are described by USP, such as the buffers on pages 3167-3168 and the gastric and intestinal fluids on page 3171.

The pharmaceutical dosage forms of the present invention are intended for oral administration to a patient in need thereof.

Certain specific aspects and embodiments of the invention will be further described in the following examples, which are provided solely for purposes of illustration and are not intended to limit the scope of the invention in any manner.

### Example 1

### Telmisartan 80 Mg and Hydrochlorothiazide 12.5 Mg Bi-Layer Tablets

Telmisartan Component:

| Ingredient | mg/Tablet |
|---|---|
| Mannitol | 320 |
| (Perlitol ™ SD 200) | |
| Sodium hydroxide | 6.6 |
| Polysorbate 80 | 3 |
| Meglumine | 24 |
| Povidone K-29/32 | 20 |
| Water* | 200 |
| Telmisartan | 80 |
| Mannitol | 38 |
| (Perlitol SD 200) | |
| Magnesium stearate | 8.4 |

Hydrochlorothiazide Component:

| Ingredient | mg/Tablet |
|---|---|
| Hydrochlorothiazide | 12.5 |
| Mannitol 25 | 150 |
| Lactose monohydrate | 29.6 |
| Iron oxide red | 0.4 |
| Povidone K-29/32 | 6 |
| Water* | 25 |
| Mannitol | 10.5 |
| (Perlitol SD 200) | |
| Magnesium stearate | 1 |

*Evaporates during processing.

Preparation of Telmisartan Component:

1. Binder solution was prepared by dissolving sodium hydroxide, polysorbate 80, meglumine and povidone K-29/32 in water.

2. Telmisartan was dissolved in the step 1 solution.

3. Granulated the mannitol (first quantity), using step 2 solution in a fluidized bed coater. Dried the granules and sized using a sieve.

4. Blended step 3 granules with mannitol (second quantity) and magnesium stearate.

Preparation of Hydrochlorothiazide Component:

1. Sifted hydrochlorothiazide, mannitol 25, lactose monohydrate and iron oxide red, and mixed.

2. Binder solution was prepared from povidone K-29/32 and water.

3. Step 1 materials were granulated using step 2 solution and the wet mass was dried in a fluidized bed drier. The dry granules were sized through a sieve.

4. Blended step 3 granules with mannitol and magnesium stearate.

Compression:

The two components were compressed into bi-layer tablets.

Dissolution testing of prepared tablets was conducted using the USP procedure and the following conditions:

For telmisartan: 900 ml of pH 7.5 phosphate buffer, USP Type II apparatus, 75 rpm stirring.

For hydrochlorothiazide: 900 ml of 0.1N HCl, USP Type II apparatus, 50 rpm stirring.

The drug dissolution data are tabulated in Table 1.

TABLE 1

| | |
|---|---|
| Time | Cumulative % of Drug Dissolved |

| (minutes) | Telmisartan | Hydrochlorothiazide |
|---|---|---|
| 10 | 52 | 52 |
| 20 | 85 | 85 |
| 30 | 96 | 96 |
| 45 | 97 | 97 |

### Examples 2-5

### Telmisartan 40 mg and Hydrochlorothiazide 12.5 mg Bi-Layer Tablets

Telmisartan Component:

| | mg/Tablet | | | |
|---|---|---|---|---|
| | Example | Example | Example | Example |
| Ingredient | 2 | 3 | 4 | 5 |
| Mannitol | 283.14 | — | — | — |
| (Perlitol SD 200) | | | | |
| Sorbitol | — | 141 | 141 | 161.35 |
| (Neosorb ™ P60W) | | | | |
| Sodium hydroxide | 3.36 | 3.1 | 3.1 | 3.1 |
| Polysorbate 80 | 1.5 | — | — | — |
| Meglumine | 12 | 10 | — | 10 |
| Povidone K-29/32 | 12 | 5.8 | 5.8 | 5.8 |
| Water* | 113.5 | 113.5 | 113.5 | 113.5 |
| Telmisartan | 40 | 40 | 40 | 40 |
| Mannitol | 40 | — | — | — |
| (Perlitol SD 200) | | | | |
| Sorbitol | — | 35.35 | 35.35 | 15 |
| (Neosorb P60W) | | | | |
| Magnesium stearate | 8 | 4.75 | 4.75 | 4.75 |

Hydrochlorothiazide Component:

| | mg/Tablet | | |
|---|---|---|---|
| | Example | Example | Examples |
| Ingredient | 2 | 3 | 4 and 5 |
| Hydrochlorothiazide | 12.5 | 12.5 | 12.5 |
| Mannitol 25 | 100 | 95 | 110 |
| Lactose monohydrate | 67.2 | 82.2 | 67.2 |
| Iron oxide red | 0.3 | 0.3 | 0.3 |
| Povidone K-29/32 | 8 | 8 | 8 |
| Water* | 25 | 20 | 20 |
| Mannitol (Perlitol SD 200) | 10 | — | — |
| Magnesium stearate | 2 | 2 | 2 |

*Evaporates during processing.

Preparation of Telmisartan Component:

1. Binder solution was prepared by dissolving sodium hydroxide, polysorbate 80, meglumine and povidone K-29/32 in water.

2. Dissolved telmisartan in the step 1 solution.

3. Granulated mannitol (first quantity) using the step 3 solution in a fluidized bed coater. Dried the granules and sized through a sieve.

4. Blended step 5 granules with mannitol (second quantity) and magnesium stearate.

Preparation of Hydrochlorothiazide Component:

1. Sifted hydrochlorothiazide, mannitol 25, lactose monohydrate and iron oxide red, and mixed.

2. Binder solution was prepared from povidone K-29/32 and water.

3. Step 1 material was granulated using step 2 solution and the wet mass was dried in a fluidized bed drier. The dry granules were sized through a sieve.

4. Blended step 3 granules with mannitol and magnesium stearate.

Compression:

The two components were compressed into bi-layer tablets.

Tablets prepared above were tested for dissolution and compared with MICARDIS PLUS (40 mg of telmisartan/12.5 mg of hydrochlorothiazide) and the data are below.

Dissolution Conditions:

A: 900 ml of 0.1N HCl, USP type II apparatus, 50 rpm stirring.

B: 900 ml of pH 7.5 phosphate buffer, USP type II apparatus, 75 rpm stirring.

| | Cumulative % of Drug Dissolved | |
|---|---|---|
| Time | Example 3 | MICARDIS PLUS |

| | | | | |
|---|---|---|---|---|
| (minutes) | Telmisartan | HCTZ | Telmisartan | HCTZ |

Medium A

| | | | | |
|---|---|---|---|---|
| 10 | 26 | 75 | 28 | 89 |
| 20 | 49 | 94 | 53 | 95 |
| 30 | 66 | 99 | 69 | 97 |
| 45 | 82 | 102 | 84 | 99 |
| 60 | 89 | 102 | 93 | 100 |

Medium B

| | | | | |
|---|---|---|---|---|
| 10 | 60 | — | 67 | — |
| 20 | 87 | — | 94 | — |
| 30 | 99 | — | 100 | — |
| 45 | 100 | — | 101 | — |
| 60 | — | — | — | — |

Prepared tablets of Example 3 and the commercial product MICARDIS PLUS (40 mg/12.5 ie 40 mg of telmisartan and 12.5 mg of hydrochlorothiazide) were packaged in blister packaging made of aluminium foil on both sides and stored at 40° C. and 75% relative humidity (RH), and at 30° C. and 65% RH, for 3 months. Samples were analyzed before, during, and after storage for moisture content (using the Karl Fischer method), impurities, dissolution and drug content. The data for 40° C. and 75% RH are in Table 2, and the data for 30° C. and 65% RH are in Table 3. In the tables, “M” is months and “ND” indicates that a substance was not detected.

TABLE 2

| | Example 3 | | | | MICARDIS PLUS | | | |
|---|---|---|---|---|---|---|---|---|
| Parameter | Initial | 1 M | 2 M | 3 M | Initial | 1 M | 2 M | 3 M |
| Moisture content (%) | — | — | — | 3 | — | 2.85 | 3.19 | 3.26 |

Impurities (% of precursor drug content)

| | | | | | | | | |
|---|---|---|---|---|---|---|---|---|
| DSA | 0.02 | 0.11 | 0.22 | 0.21 | 0.12 | 0.13 | 0.14 | 0.23 |
| Chlorothiazide | ND | ND | ND | ND | ND | 0.03 | 0.02 | 0.04 |
| Highest unidentified | 0.1 | 0.06 | 0.06 | 0.06 | 0.02 | 0.02 | ND | 0.02 |
| impurity | | | | | | | | |
| Total impurities | 0.12 | 0.17 | 0.28 | 0.27 | 0.16 | 0.18 | 0.16 | 0.34 |

Dissolution (% of contained drug)*

| | | | | | | | | |
|---|---|---|---|---|---|---|---|---|
| Telmisartan (45 minutes) | 99 | 103 | 110 | 95 | — | 92 | 95 | 101 |
| Hydrochlorothiazide (60 | 95 | 96 | 89 | 96 | — | 99 | 101 | 100 |
| minutes) | | | | | | | | |

Assay (% of label dose)

| | | | | | | | | |
|---|---|---|---|---|---|---|---|---|
| Telmisartan | 99.2 | 103 | 105 | 103 | 96.8 | 97.5 | 97.4 | 99.7 |
| Hydrochlorothiazide | 97.5 | 96.3 | 96.9 | 96 | 98.7 | 99.2 | 98 | 99 |

TABLE 3

| | Example 3 | | | | MICARDIS PLUS | | | |
|---|---|---|---|---|---|---|---|---|
| Parameter | Initial | 1 M | 2 M | 3 M | Initial | 1 M | 2 M | 3 M |
| Moisture content (%) | — | — | — | 3.56 | — | 2.76 | 3.06 | — |

Impurities (% of precursor drug content)

| | | | | | | | | |
|---|---|---|---|---|---|---|---|---|
| DSA | 0.02 | 0.04 | 0.05 | 0.1 | 0.12 | 0.15 | 0.11 | — |
| Chlorothiazide | ND | ND | ND | ND | ND | 0.04 | 0.03 | — |
| Highest unidentified impurity | 0.1 | 0.06 | 0.06 | 0.06 | 0.02 | 0.02 | ND | — |
| Total impurities | 0.12 | 0.1 | 0.11 | 0.16 | 0.16 | 0.2 | 0.13 | — |

Dissolution (% of contained drug)*

| | | | | | | | | |
|---|---|---|---|---|---|---|---|---|
| Telmisartan (45 minutes) | 99 | 102 | 102 | 104 | — | 92 | 95 | — |
| Hydrochlorothiazide (60 | 95 | 96 | 92 | 93 | — | 97 | 100 | — |
| minutes) | | | | | | | | |

Assay (% of label dose)

| | | | | | | | | |
|---|---|---|---|---|---|---|---|---|
| Telmisartan | 99.2 | 102 | 105 | 103 | 96.8 | 97.6 | 97.3 | — |
| Hydrochlorothiazide | 97.5 | 97.3 | 97.4 | 95.7 | 98.7 | 98.7 | 97.2 | — |

*Dissolution conditions: 900 ml of pH 7.5 phosphate buffer, USP type II apparatus, 75 rpm stirring, 37° C. ± 0.5° C.

### Example 6

### Telmisartan 80 mg and Hydrochlorothiazide 12.5 mg Bi-Layer Tablets

Telmisartan Component:

| Ingredient | mg/Tablet |
|---|---|
| Microcrystalline | 320 |
| cellulose | |
| (Avicel ™ PH101) | |
| Sodium hydroxide | 6.6 |
| Polysorbate 80 | 3 |
| Meglumine | 24 |
| Povidone K-29/32 | 20 |
| Water* | 200 |
| Telmisartan | 80 |
| Mannitol | 38 |
| (Perlitol SD 200) | |
| Magnesium stearate | 8.4 |

Hydrochlorothiazide Component:

| Ingredient | mg/Tablet |
|---|---|
| Hydrochlorothiazide | 12.5 |
| Mannitol 25 | 150 |
| Lactose monohydrate | 29.6 |
| Iron oxide red | 0.4 |
| Povidone K-29/32 | 6 |
| Water* | 25 |
| Mannitol | 10.5 |
| (Perlitol SD 200) | |
| Magnesium stearate | 1 |

*Evaporates during processing.

Preparation of Telmisartan Component:

1. Binder solution was prepared by dissolving sodium hydroxide, polysorbate 80, meglumine and povidone K-29/32 in water.

2. Dissolved telmisartan in step 1 solution.

3. Granulated the microcrystalline cellulose using step 2 solution in a fluidized bed coater. Dried the granules and sized by passing through a sieve.

4. Blended step 3 granules with mannitol and magnesium stearate.

Preparation of Hydrochlorothiazide Component:

1. Sifted hydrochlorothiazide, mannitol 25, lactose monohydrate and iron oxide red and mixed.

2. Binder solution was prepared by using povidone K-29/32 and water.

3. Step 1 material was granulated using step 2 solution and the wet mass was dried in a fluidized bed drier. The dry granules were sized using a sieve.

4. Blended step 3 granules with mannitol and magnesium stearate.

Compression:

The telmisartan and hydrochlorothiazide components were compressed into bi-layer tablets.

### Examples 7-10

### Telmisartan 80 mg and Hydrochlorothiazide 12.5 mg Bi-Layer Tablets

Telmisartan Component:

| | mg/Tablet | | | | |
|---|---|---|---|---|---|
| | | | | Example | Example |
| Ingredient | Example 7 | Example 8 | Example 9 | 10 | 11 |
| Microcrystalline | 320 | — | — | | — |
| cellulose (Avicel PH101) | | | | | |
| Sorbitol (Neosorb P60W) | — | 322.7 | 282 | 300 | 282 |
| Sodium hydroxide | 6.6 | 6.2 | 6.2 | 6.2 | 6.2 |
| Polysorbate 80 | 3 | — | — | | — |
| Meglumine | 24 | 20 | 20 | 20 | 20 |
| Povidone K-29/32 | 20 | 11.6 | 11.6 | 5.8 | 11.6 |
| Water* | 200 | 227 | 227 | 227 | 227 |
| Telmisartan | 80 | 80 | 80 | 80 | 80 |
| Mannitol (Perlitol SD 200) | 38 | — | — | | — |
| Sorbitol (Neosorb P60W) | | 30 | 70.7 | 48.5 | 70.7 |
| Magnesium stearate | 8.4 | 9.5 | 9.5 | 9.5 | 9.5 |

Hydrochlorothiazide Component:

| | mg/Tablet | | | |
|---|---|---|---|---|
| | Example | Example | Examples | Example |
| Ingredient | 7 | 8 | 9 and 10 | 11 |
| Hydrochlorothiazide | 12.5 | 12.5 | 12.5 | 12.5 |
| Mannitol 25 | 150 | 110 | 95 | 110 |
| Lactose monohydrate | — | 67.2 | 82.2 | 67.2 |
| Microcrystalline | 29.6 | — | | — |
| cellulose | | | | |
| (Avicel PH101) | | | | |
| Iron oxide red | 0.4 | 0.3 | 0.3 | 0.3 |
| Povidone K-29/32 | 6 | 8 | 8 | 8 |
| Water* | 25 | 20 | 20 | 20 |
| Mannitol | 10.5 | — | — | — |
| (Perlitol SD 200) | | | | |
| Magnesium stearate | 1 | 2 | 2 | 2 |

*Evaporates during processing.

Preparation of Telmisartan Component:

1. Binder solution was prepared by dissolving sodium hydroxide, polysorbate 80, meglumine and povidone K-29/32 in water.

2. Dissolved telmisartan in step 1 solution.

3. Granulated the microcrystalline cellulose using step 2 solution in a fluidized bed coater. Dried the granules and sized by passing through a sieve.

4. Blended step 3 granules with mannitol and magnesium stearate.

Preparation of Hydrochlorothiazide Component:

1. Sifted hydrochlorothiazide, microcrystalline cellulose, mannitol and iron oxide red, and mixed.

2. Binder solution was prepared by adding povidone K-29/32 to water.

3. Step 1 material was granulated using step 2 solution and the wet mass was dried in a fluidized bed drier. The dry granules were sized through a sieve.

4. Blended step 3 granules with mannitol and magnesium stearate.

Compression:

The telmisartan and hydrochlorothiazide components were compressed into bi-layer tablets.

The tablets of Example 8 were evaluated for hardness (expressed in kiloponds, kp), friability (%), and content uniformity of telmisartan and hydrochlorothiazide. The data are in Table 4.

TABLE 4

| Parameter | Example 8 |
|---|---|
| Hardness (kp) | 11 |
| Friability (%) | 0.15 |
| Content uniformity | Minimum 99 |
| (Telmisartan layer) | Maximum 103 |
| | Relative standard |
| | deviation (RSD) 1.22 |
| Content uniformity | Minimum 99 |
| (Hydrochlorothiazide | Maximum 101.7 |
| layer) | RSD 0.92 |

Dissolution Testing*

| | |
|---|---|
| Time | Cumulative % of Drug Dissolved |

| (minutes) | Telmisartan | Hydrochlorothiazide |
|---|---|---|
| 10 | 38 | 85 |
| 20 | 67 | 100 |
| 30 | 87 | 103 |
| 45 | 101 | 103 |

*900 mL of pH 7.5 phosphate buffer, 75 RPM stirring, USP 2 apparatus.

Tablets of Example 9 and the commercial product MICARDIS PLUS (80 mg telmisartan/12.5 mg hydrochlorothiazide) were packaged in blister packaging made of aluminium foil on both sides and stored at 40° C. and 75% RH, and at 30° C. and 65% RH, for 3 months. Samples were analyzed for moisture content, impurities, dissolution and drug content, before, during, and after the storage. The data for 40° C. and 75% RH are in Table 5 and the data for 30° C. and 65% RH are in Table 6, where “M” in the tables is months and “ND” means a substance was not detected.

TABLE 5

| | Example 9 | | | | MICARDIS PLUS | | | |
|---|---|---|---|---|---|---|---|---|
| Parameter | Initial | 1 M | 2 M | 3 M | Initial | 1 M | 2 M | 3 M |
| Moisture content (%) | — | 3.06 | — | 3.43 | — | 2.41 | 2.67 | 2.84 |

Impurities (% of precursor drug content)

| | | | | | | | | |
|---|---|---|---|---|---|---|---|---|
| DSA | 0.02 | 0.28 | 0.35 | 0.47 | 0.12 | 0.13 | 0.13 | 0.18 |
| Chlorothiazide | ND | ND | ND | ND | ND | 0.03 | 0.03 | 0.07 |
| Highest unidentified | 0.16 | 0.05 | 0.06 | 0.06 | 0.05 | 0.03 | 0.03 | 0.07 |
| impurity | | | | | | | | |
| Total impurities | 0.27 | 0.33 | 0.41 | 0.53 | 0.26 | 0.2 | 0.23 | 0.26 |

Dissolution (% of contained drug)*

| | | | | | | | | |
|---|---|---|---|---|---|---|---|---|
| Telmisartan (45 minutes) | 104 | 91 | 99 | 100 | 98 | 100 | 100 | 102 |
| Hydrochlorothiazide (60 | 98 | 102 | 104 | 106 | 99 | 98 | 96 | 101 |
| minutes) | | | | | | | | |

Assay (% of label dose)

| | | | | | | | | |
|---|---|---|---|---|---|---|---|---|
| Telmisartan | 99.3 | 98.8 | 97.3 | 99.7 | 98.7 | 98.9 | 98.1 | 100.5 |
| Hydrochlorothiazide | 101 | 99 | 100 | 99 | 98.7 | 98.6 | 98.5 | 98.1 |

TABLE 6

| | Example 9 | | | | MICARDIS PLUS | | | |
|---|---|---|---|---|---|---|---|---|
| Parameter | Initial | 1 M | 2 M | 3 M | Initial | 1 M | 2 M | 3 M |
| Moisture content (%) | — | — | — | 3.41 | — | 2.38 | 2.86 | — |

Impurities (% of precursor drug content)

| | | | | | | | | |
|---|---|---|---|---|---|---|---|---|
| DSA | 0.02 | 0.06 | 0.13 | 0.2 | 0.12 | 0.12 | 0.11 | — |
| Chlorothiazide | ND | ND | ND | ND | ND | ND | 0.03 | — |
| Highest unidentified | 0.16 | 0.05 | 0.06 | 0.06 | 0.05 | 0.02 | ND | — |
| impurity | | | | | | | | |
| Total impurities | 0.27 | 0.11 | 0.19 | 0.26 | 0.26 | 0.16 | 0.14 | — |

Dissolution (% of contained drug)*

| | | | | | | | | |
|---|---|---|---|---|---|---|---|---|
| Telmisartan (45 minutes) | 104 | 97 | 99 | 105 | 98 | 106 | 100 | — |
| Hydrochlorothiazide (60 | 98 | 102 | 102 | 104 | 99 | 97 | 92 | — |
| minutes) | | | | | | | | |

Assay (% of label dose)

| | | | | | | | | |
|---|---|---|---|---|---|---|---|---|
| Telmisartan | 99.3 | 97.1 | 98.6 | 98.6 | 98.7 | 98.3 | 98 | — |
| Hydrochlorothiazide | 101 | 98.6 | 101 | 100 | 98.7 | 97.6 | 98 | — |

*Dissolution conditions: 900 ml of pH 7.5 phosphate buffer, USP type II apparatus, 75 rpm stirring, 37° C. ± 0.5° C.

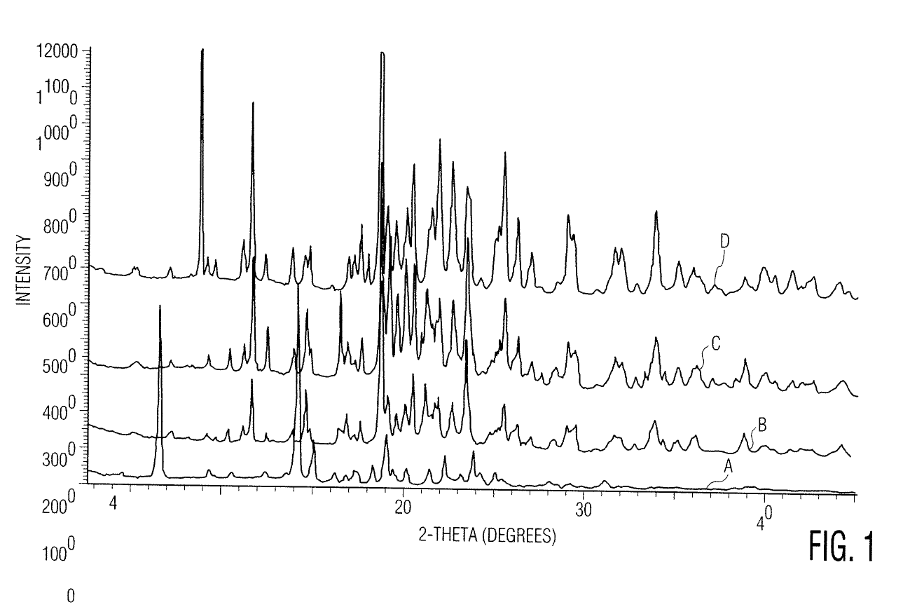

Tablets of Example 10 were packaged in blister packaging made of aluminium foil on both sides and stored at 40° C. and 75% RH for 1 month. FIG. 1 is a comparison of powder X-ray diffraction (XRD) patterns, using copper Kα-1 radiation, of the telmisartan ingredient (A), the formulation as originally prepared (B), and the formulation after storage (C). A “placebo” formulation was similarly prepared using the above ingredients, but omitting the telmisartan, and the XRD pattern of the placebo is also shown (P). The XRD pattern of the formulation before storage matches that of the stored formulation, showing polymorphic stability.

### Example 12

### Dissolution and Pharmacokinetics Study

Tablets from Example 9 were tested for dissolution and compared with MICARDIS PLUS (80 mg of telmisartan/12.5 mg of hydrochlorothiazide) and the data are below:

Dissolution Conditions:

A: 900 ml of 0.1N HCl, USP type II apparatus, 50 rpm stirring.

B: 900 ml of pH 7.5 phosphate buffer, USP type II apparatus, 75 rpm stirring.

| | Cumulative % of Drug Dissolved | |
|---|---|---|
| Time | Example 9 | MICARDIS PLUS |

| | | | | |
|---|---|---|---|---|
| (minutes) | Telmisartan | HCTZ | Telmisartan | HCTZ |

Medium A

| | | | | |
|---|---|---|---|---|
| 10 | 17 | 85 | 19 | 74 |
| 20 | 36 | 96 | 36 | 91 |
| 30 | 49 | 99 | 48 | 96 |
| 45 | 62 | 100 | 62 | 99 |
| 60 | 72 | 104 | 74 | 99 |

Medium B

| | | | | |
|---|---|---|---|---|
| 10 | 42 | — | 46 | — |
| 20 | 67 | — | 69 | — |
| 30 | 82 | — | 86 | — |
| 45 | 96 | — | 98 | — |
| 60 | — | — | — | — |

Tablets were further evaluated in a two way crossover bioequivalence study involving administration of the tablets of Example 9 as a test product (“T”) and the commercial product MICARDIS PLUS (80 mg of telmisartan and 12.5 mg of hydrochlorothiazide) as a reference product (“R”) to 16 healthy human volunteers in a fasted state, and plasma concentrations of the drug compounds were determined at intervals after dosing.

The following parameters were calculated:

AUC₀₋ₜ=the area under plasma concentration versus time curve, from the time of administration to the last measurable concentration.

AUC_{0-∞}=area under the plasma concentration versus time curve, from the time of administration to infinity.

Cₘₐₓ=maximum plasma concentration.

Tₘₐₓ=time after administration until the maximum measured plasma concentration.

The results of these pharmacokinetic parameters for each drug were calculated and are summarized in the following table:

| | | | |
|---|---|---|---|
| | Example | Reference | Ratio |
| Parameter | 11 (T) | (R) | (T ÷ R × 100) |

Telmisartan

| | | | |
|---|---|---|---|
| AUC₀₋ₜ (ng · hour/mL) | 2442.06 | 2452.45 | 97.18 |
| AUC_{0-∞} (ng · hour/mL) | 2486.27 | 2490.51 | 97.59 |
| Cₘₐₓ (ng/mL) | 461.12 | 405.17 | 106.8 |
| Tₘₐₓ (hours) | 1 | 1.33 | — |

Hydrochlorothiazide

| | | | |
|---|---|---|---|
| AUC₀₋ₜ (ng · hour/mL) | 574.93 | 545.3 | 105.4 |
| AUC_{0-∞} (ng · hour/mL) | 582.59 | 551.62 | 105.6 |
| Cₘₐₓ (ng/mL) | 80.51 | 72.86 | 110.8 |
| Tₘₐₓ (hours) | 1.67 | 2 | — |

### Example 13

### Telmisartan 80 mg and Hydrochlorothiazide 12.5 mg Multi-Particulate Tablets

Telmisartan Component:

| Ingredient | mg/Tablet |
|---|---|
| Sugar spheres | 347 |
| Telmisartan | 80 |
| Sodium hydroxide | 6.2 |
| Meglumine | 20 |
| Povidone K-29/32 | 5.8 |
| Isopropyl alcohol* | 60 |
| Water* | 90 |
| Hydroxypropyl | 9.4 |
| methylcellulose | |
| Polyethylene glycol | 1 |
| Isopropyl alcohol* | 18 |
| Dichloromethane* | 12 |

Hydrochlorothiazide Component:

| Ingredient | mg/Tablet |
|---|---|
| Hydrochlorothiazide | 12.5 |
| Mannitol 25 | 150 |
| Lactose monohydrate | 29.6 |
| Iron oxide red | 0.4 |
| Povidone K-29/32 | 6 |
| Water* | 25 |
| Mannitol | 10.5 |
| (Perlitol SD 200) | |
| Magnesium stearate | 1 |

*Evaporates during processing.

Preparation of Telmisartan Component:

1. Binder solution was prepared by dissolving sodium hydroxide, polysorbate-80, meglumine and povidone K-29/32 in a mixture of water and isopropyl alcohol (first quantity).

2. Dissolved telmisartan in step 1 solution.

3. Sugar spheres were loaded into a fluidized bed coater and the solution of step 2 was applied by spraying.

4. Hydroxypropyl methylcellulose was dispersed in isopropyl alcohol (second quantity).

5. Polyethylene glycol was dissolved in dichloromethane and added to step 4 dispersion.

5. Drug loaded pellets of step 3 were coated with step 5 coating solution in the fluidized bed coater.

Preparation of Hydrochlorothiazide Component:

1. Sifted hydrochlorothiazide, mannitol 25, lactose monohydrate and iron oxide red, and mixed.

2. Binder solution was prepared by adding povidone K-29/32 to water.

3. Step 1 material was granulated using step 2 solution and the wet mass was dried in a fluidized bed drier. The dry granules were sized through a sieve.

Blending and Compression:

The telmisartan pellets and hydrochlorothiazide granules were blended with mannitol and magnesium stearate and compressed into tablets.

### Example 14

### Telmisartan 80 mg and Hydrochlorothiazide 12.5 mg Multi-Particulate Tablets, Hydrochlorothiazide Coated onto Telmisartan Core Tablet

Telmisartan in Dissolving Matrix:

| Ingredient | mg/Tablet |
|---|---|
| Mannitol | 348.5 |
| Sodium hydroxide | 6.2 |
| Meglumine | 20 |
| Povidone K-29/32 | 5.8 |
| Water* | 60 |
| Isopropyl alcohol* | 90 |
| Telmisartan | 80 |
| Magnesium stearate | 9.5 |

Hydrochlorothiazide Component:

| Ingredient | mg/Tablet |
|---|---|
| Hydrochlorothiazide | 12.5 |
| Hydroxypropyl | 10.5 |
| methylcellulose | |
| Polyethylene glycol | 1 |
| Isopropyl alcohol* | 15 |
| Dichloromethane* | 5 |

*Evaporates during processing.

Telmisartan-Containing Cores:

1. Binder solution was prepared by dissolving sodium hydroxide, meglumine and povidone K-29/32 in a mixture of water and isopropyl alcohol.

2. Telmisartan was dissolved in step 1 solution.

3. Mannitol was granulated using step 2 drug solution in fluidized bed coater.

4. Dried granules were sieved.

5. Granules of step 4 were blended with magnesium stearate.

6. The step 5 granules were compressed into tablets using a tablet compression machine.

Hydrochlorothiazide Component:

1. Hydroxypropyl methylcellulose and hydrochlorothiazide were dissolved in isopropyl alcohol.

2. Polyethylene glycol was dissolved in dichloromethane.

3. Step 1 and step 2 were mixed together to form a coating solution.

Coating:

The telmisartan tablets were loaded into a coating machine and hydrochlorothiazide coating solution was applied.

### Example 15

### Telmisartan 80 mg and Hydrochlorothiazide 12.5 mg Multi-Particulate Tablet, Hydrochlorothiazide Coated onto Telmisartan Core Tablet

Telmisartan in Disintegrating Matrix:

| Ingredient | mg/Tablet |
|---|---|
| Microcrystalline | 348.5 |
| cellulose | |
| Sodium hydroxide | 6.2 |
| Meglumine | 20 |
| Povidone K-29/32 | 5.8 |
| Water* | 60 |
| Isopropyl alcohol* | 90 |
| Telmisartan | 80 |
| Magnesium stearate | 9.5 |

Hydrochlorothiazide Component:

| Ingredient | mg/Tablet |
|---|---|
| Hydrochlorothiazide | 12.50 |
| Hydroxypropyl | 10.50 |
| methylcellulose | |
| Polyethylene glycol | 1.00 |
| Isopropyl alcohol* | 15 |
| Dichloromethane* | 5 |

*Evaporates during processing.

Telmisartan-Containing Cores:

1. Binder solution was prepared by dissolving sodium hydroxide, meglumine and povidone K-29/32 in a mixture of water and isopropyl alcohol.

2. Telmisartan was dissolved in step 1 solution.

3. Microcrystalline cellulose was granulated using step 2 drug solution in a fluidized bed coater.

4. Dried granules were sieved.

5. Granules of step 4 were blended with magnesium stearate.

6. Step 5 granules were compressed into tablets using a tablet compression machine.

Hydrochlorothiazide Component:

1. Hydroxypropyl methylcellulose and hydrochlorothiazide were dissolved in isopropyl alcohol.

2. Polyethylene glycol was dissolved in dichloromethane

3. Step 1 and step 2 were mixed together to form a coating solution.

Coating:

Telmisartan-containing tablets were loaded into a tablet coating machine and hydrochlorothiazide coating solution was applied.

1. A pharmaceutical formulation comprising a first portion containing telmisartan in a dissolving matrix and a second portion containing hydrochlorothiazide in a dissolving matrix.

2. The pharmaceutical formulation of claim 1, in the form of a bi-layer tablet.

3. The pharmaceutical formulation of claim 1, in the form of a particle comprising telmisartan, coated with a composition comprising hydrochlorothiazide.

4. The pharmaceutical formulation of claim 3, wherein a particle is a tablet.

5. The pharmaceutical formulation of claim 1 wherein a dissolving matrix does not contain a disintegrant ingredient.

6. The pharmaceutical formulation of claim 1 containing a DSA impurity at less than about 1 percent by weight of the initial hydrochlorothiazide content.

7. The pharmaceutical formulation of, claim 1 containing drug compound-derived total impurities at less than about 4 percent by weight of the initial total telmisartan and hydrochlorothiazide content.

8. The pharmaceutical formulation of, claim 1 containing less than about 6 percent by weight of moisture.

9. The pharmaceutical formulation of, claim 1 producing plasma telmisartan cₘₐₓ values about 350 ng/mL to about 600 ng/mL, and AUC₀₋ₜ values about 1950 ng·hour/mL to about 3100 ng·hour/mL, after oral administration of a single 80 mg telmisartan dose to healthy humans.

10. The pharmaceutical formulation of, claim 1 producing plasma hydrochlorothiazide Cₘₐₓ values about 60 ng/mL to about 100 ng/mL, and AUC₀₋ₜ values about 450 ng·hour/mL to about 720 ng·hour/mL, after oral administration of a single 12.5 mg hydrochlorothiazide dose to healthy humans.

11. The pharmaceutical formulation of, claim 1 producing at least one of:
i) a Tₘₐₓ value about 0.3 hours to about 2 hours for telmisartan; and
ii) a Tₘₐₓ value about 1 hour to about 3 hours for hydrochlorothiazide;
after oral administration of a single dose to healthy humans.

12. A process for preparing a pharmaceutical bi-layer tablet comprising:
a) granulating a solid composition comprising a diluent with a solution comprising telmisartan, to provide a first tablet layer composition;
b) combining hydrochlorothiazide with at least one solid pharmaceutical excipient to provide a second tablet layer composition; and
c) compressing a first tablet layer composition and a second tablet layer composition into a bi-layer tablet.

13. The process of claim 12, wherein b) further comprises granulating a combination comprising hydrochlorothiazide and a solid pharmaceutical excipient with a binder solution.
